(19) United States
(12) Patent Application Publication
(10) Pub. No.: US 2020/0016062 A1
(43) Pub. Date: Jan. 16, 2020

(54) TERMINALIA FERDINANDIANA LEAF EXTRACT AND PRODUCTS CONTAINING EXTRACT OF TERMINALIA FERDINANDIANA LEAF

(71) Applicant: RISING PHOENIX INDUSTRIES PTY LTD, MOUNT LAWLEY, WA (AU)

(72) Inventors: Ian Edwin Cock, Logan Central (AU); David John Boehme, Humpty Doo (AU); Roslyn Anne Miles, Myaree (AU)

(21) Appl. No.: 16/470,960

(22) PCT Filed: Dec. 18, 2017
(86) PCT No.: PCT/AU2017/051406

§ 371 Date: Jun. 18, 2019

(30) Foreign Application Priority Data
Dec. 20, 2016 (AU) 2016905274

(51) Int. Cl. A61K 8/9789; A61Q 15/00; A61K 8/49; A61Q 19/08; A61Q 17/00; A61Q 17/04; A61K 8/04

(52) U.S. Cl. CPC A61K 8/9789; A61Q 15/00; A61K 8/498; A61K 8/042; A61Q 17/005; A61Q 17/04; A61Q 19/08

(57) ABSTRACT

Terminalia ferdinandiana (T. ferdinandiana) leaf extract can be provided in a composition for use in inhibiting bacterial growth. The extract or composition can be in or as a personal hygiene product e.g. a deodorant or anti-perspirant, a cosmetic, a cosmeceutical, a surface treatment product or a preservative, and can include T. ferdinandiana fruit extract. The leaf extract can be a methanolic extract, alcoholic extract, steam extract, aqueous extract; ethyl acetate extract; chloroform extract; or hexane extract, and can include a tannin e.g. chebulic acid, corilagen, chebulinic acid or chebulagic acid and/or at least one flavone e.g. luteolin. The composition can be provided as a topical skin application.

### FIELD OF THE INVENTION

The present invention relates to natural extracts/derivatives of Terminalia ferdinandiana (T. ferdinandiana).

### BACKGROUND TO THE INVENTION

Hereinafter, Terminalia ferdinandiana may be referred to as T. ferdinandiana for ease of reference.

T. ferdinandiana is a small, deciduous tree which grows wild extensively throughout the subtropical woodlands of northern tracts of Australia, typically in the Northern Territory and Western Australia.

T. ferdinandiana bears an abundant crop of small plum-like fruits. The fruit is known to have very high vitamin C content, and is a source of antioxidants, folic acid and iron. The fruit and extracts of the fruit are used in foods, dietary supplements and pharmaceuticals.

The commonest use of T. ferdinandiana fruits is for gourmet jams, sauces, juices, ice-cream, cosmetics, flavours and pharmaceuticals.

Cosmetic use of T. ferdinandiana fruit has been disclosed in European patent publication number EP 1981513. EP 1581513 discloses cosmetic methods for use in beneficial skin applications of T. ferdinandiana fruit and/or acai berry extract.

Examples of cosmetic vehicles for the T. ferdinandiana fruit extract proposed in EP 1581513 include emulsions (e.g. oil-in-water and water-in-oil emulsions), creams, lotions, solutions (e.g. aqueous or hydro-alcoholic solutions), anhydrous bases (e.g. lipstick or a powder), gels, and ointments. EP 1581513 also proposes that extract of T. ferdinandiana fruit be included in anti-aging, cleansing, or moisturizing products.

Another U.S. Pat. No. 7,175,862 discloses a method of producing a powder containing ascorbic acid (vitamin C), antioxidants and phytochemicals from the fruit of the T. ferdinandiana plant.

U.S. Pat. No. 7,175,862 discusses use of the powdered T. ferdinandiana fruit for the reduction of free radicals in the human body. Free radicals are chemicals understood to cause damage to tissue, cells, fat and proteins that come into contact with the free radicals. Oxygen, although an essential chemical of life, is one of the leading producers of free radicals in the body that causes oxidative damage. This damage has been linked to cardiovascular diseases, cataracts and DNA damage, which may lead to development of cancer or contribute to accelerated aging.

Vitamin C is understood to reduce the effects of free radicals on human tissue. The high vitamin C content of the T. ferdinandiana fruit has therefore become a preferred alternative over orange, lemon and grapefruit sources for some producers. The T. ferdinandiana fruit has been found to contain typically 3000 mg of vitamin C per 100 g of fruit—over 50 times the concentration of vitamin C found in oranges.

Deodorants are often alcohol based. Alcohol initially stimulates sweating, but may also temporarily kill bacteria.

Other active ingredients in modern deodorant sprays, roll-ons and solid stick deodorants include sodium stearate, sodium chloride and stearyl alcohol. These all contain synthetically originating active chemical constituents.

Deodorants can be formulated with other, more persistent antimicrobials such as triclosan that slow bacterial growth or with metal chelant compounds such as EDTA. Deodorants may contain perfume fragrances or natural essential oils intended to mask the odor of perspiration. In the past, deodorants included chemicals such as zinc oxide, acids, ammonium chloride, sodium bicarbonate and formaldehyde, but some of these ingredients were messy, irritating to the skin or even carcinogenic.

Triclosan, first introduced as a pesticide, is a high production volume ingredient used as a bactericide in personal care products such as toothpaste, deodorant, and antibacterial soap, and also integrated into furniture, toys and plastics used for food preparation, has been linked to heart disease and heart failure.

In September 2016, the USA Federal Drug Administration (FDA) announced that effective September 2017, it would prohibit the sale of “consumer antiseptic washes” containing triclosan or 18 other ingredients marketed as antimicrobials due to the FDA's findings of the lack of efficacy in these products.

As of 2013 the FDA found clear benefit to health for some consumer products containing triclosan but not in others; for example the FDA had no evidence that triclosan in antibacterial soaps and body washes provides any benefit over washing with regular soap and water.

Aluminium is a common additive in modern deodorants despite the widely accepted negative health effects of aluminium, such as Alzheimer's disease.

Over-the-counter products labeled as “natural deodorant crystal” containing the chemical potassium alum, which contains aluminium, have gained some popularity as an alternative ‘health product’, in spite of concerns about possible contact dermatitis.

An alternative to modern commercial deodorants is ammonium alum, which is a common type of alum, also containing aluminium, sold in crystal form and often referred to as a deodorant crystal.

It will be appreciated that body malodour is generally socially unacceptable, potentially resulting in social exclusion of an individual and resulting emotional and personal confidence consequences.

In particular production of excessive axillary (relating to the armpit) and foot odours relates to excessive sweat production, which has economic consequences for an individual in respect of replacing stained and smelly clothing and the need to purchase numerous or large quantities of (antiperspirant) deodorant.

Corynebacterium spp. possess enzymes to degrade protein and lipid sweat components to produce strongly malodorous volatile components. Staphylococcus spp. possess similar (though less active) enzymes and are therefore capable of producing similar volatile compounds and therefore also contribute to axillary malodour.

Propionbacterium spp. and Brevibacterium are the main bacterial contributors to foot malodour production.

Investigation of the T. ferdinandiana plant has realised hitherto unrealised beneficial properties of the T. ferdinandiana plant that addresses one or more of the shortcomings of the aforementioned synthetic or naturally derived products.

### SUMMARY OF THE INVENTION

With the aforementioned in mind, an aspect of the present invention provides a composition for use in inhibiting presence of bacteria, the composition including extract of T. ferdinandiana leaf.

A further aspect of the present invention provides a cosmetic method for inhibiting bacterial presence on skin, the method comprising applying a skin care composition comprising T. ferdinandiana leaf extract to the skin.

Inhibiting presence of bacteria may include one or more of preventing bacterial growth or reducing presence of bacteria already present.

Preferably the application to the skin includes topical application of the composition, such as of a cream, lotion or gel.

The composition may be provide as, or included in, a deodorant, such as a body deodorant for personal hygiene purposes.

More preferably, the topically applied composition may be, or may be included in, a direct body contact product, such as a deodorant, a soap, a toothpaste, a hand and/or body wash, hair shampoo. The composition may be provided as or in a skin lightening product. For example, the composition may be added to an existing skin lightening product.

Preferably the composition is provided in a personal hygiene or cosmetic product as a preservative for prolonging or extending the efficacy and/or storage life of such products.

Many cosmetics and personal hygiene products are thrown away because they have deteriorated and spoiled over time. This wastes product and results in more packaging going into landfill. It would be beneficial to prolong the shelf life/storage life of such cosmetics and personal hygiene products to reduce wastage and packaging going into landfill.

The composition may be provided in a product as an anti-bacterial agent to ameliorate deterioration of cosmetic and/or personal hygiene products. Bacteria are often transferred to the product from touching the product with your hands, such as in moisturiser or foundation products used to touch the skin, particularly if the product is applied with an inbuilt applicator, such as a pad or roller-ball, or is applied by direct contact with the skin.

The composition may be present as an anti-bacterial in a cosmetic product, such as a moisturiser, mascara, foundation, powder, lipstick or skin tint lotion/cream/gel.

The composition may be deployed via a direct body contact applicator, such as a roll-on or solid product, or by a non-contact applicator, such as a spray. The composition may be entrained in a spray from a pressurised or non-pressurised container.

The composition may be present in a carrier fluid, such as a gel, lotion, liquid or cream, and may be delivered from a manually pumped or pressurised aerosol type container.

Preferably, one or more embodiments of the present invention is effective in reducing or preventing malodour arising from bacteria breaking down body sweat by controlling the activity and/or presence of bacteria.

One or more embodiments of the present invention may be provided as or in an antiperspirant product for personal hygiene, such as a roll on lotion, aerosol spray or solid stick product.

Preferably the leaf extract includes one or more of a methanolic extract, aqueous extract; ethyl acetate extract; chloroform extract; or hexane extract of the T. ferdinandiana leaf.

Preferably the T. ferdinandiana leaf extract includes a proportion of at least one tannin. More preferably the at least one tannin includes one or more of chebulic acid, corilagen, chebulinic acid and chebulagic acid.

The T. ferdinandiana leaf extract may preferably include at least one flavone or flavonoid, such as luteolin.

Furthermore, the T. ferdinandiana leaf extract may include at least one anti-oxidant, such as an ellagic acid e.g. ellagic acid dehydrate and/or trimethyl ellagic acid.

A further aspect of the present invention provides a method of inhibiting growth of bacteria includes applying a composition containing an extract of Terminalia ferdinandiana leaf to a surface having bacteria thereon or a surface at risk of having bacteria thereon. The surface may be or include human skin and the composition is applied to the skin.

Another aspect of the present invention provides a composition for use in inhibiting presence of bacteria on surfaces, the composition including extract of T. ferdinandiana leaf.

The composition may be utilised as a spray product, a pour on liquid product, a wash, a dilutable liquid concentrate, a wipe on product or impregnated into a material for use as a wipe.

Another aspect of the present invention provides an extract of Terminalia ferdinandiana leaf for use in or as a bactericide or antibacterial product.

The extract or composition may be applied to one or more axillary regions and/or at least one foot of a person.

The extract or composition may be applied by spray or application of a gel or cream.

Preferably the extract of Terminalia ferdinandiana leaf is mixed into a carrier product such that the Terminalia ferdinandiana leaf extract act as an active antibacterial agent. One or more other antibacterial agents may be included in the carrier product. The carrier product may be a liquid, cream, gel, solid, and may be delivered by spray, pouring, impregnated into a wipe, sponge or tissue, and may be provided as a sterile medical product provided in a sealed package preventing exposure to the atmosphere until opened.

Another aspect of the present invention provides an extract of T. ferdinandiana including extract of T. ferdinandiana leaf.

Preferably the extract is for use as a bacteria inhibition composition.

The extract may further include extract of T. ferdinandiana fruit.

The extract of T. ferdinandiana according to claim 22 or 23, including at least one tannin or at least one flavone.

The at least one tannin may include one of or a combination of two or more of, chebulic acid, corilagen, chebulinic acid and chebulagic acid. Alternatively, or in addition, the extract may include at least one flavone or flavinoid, such as luteolin.

The extract may include one or more anti-oxidants, such as an ellagic acid e.g. ellagic acid dehydrate and/or trimethyl ellagic acid.

It will be appreciated that one or more forms of the present invention may be, or may be included or incorporated into, one or more of the following products: a liquid cleanser, anti-bacterial additive for liquids, antimicrobial preservative for cosmetics, antimicrobial preservative for pharmaceuticals, personal hygiene products, cosmetics as a preservative or anti-bacterial agent, cleanser for the face; cosmetic acne cleanser, facial cleanser, hand cleanser, detergent having disinfectant properties, disinfectant soap, disinfectant soap solution, detergent having disinfectant properties, antiperspirant deodorant, body deodorant, deodorant cream for personal use, deodorant preparation for personal use, deodorant soap; deodorant spray for personal use, deodorant sticks for personal use, deodorant for human beings or for animals, deodorant for personal use; deodorant for pets; personal body deodorant, foot deodorant, douching preparation for personal sanitary or deodorant purposes, essential oils for use in deodorants, perfumed deodorant for personal use, roll-on deodorant, scented deodorant preparations for personal use, deodorant for clothing and textiles, deodorant for household use, perfumed deodorant, room air freshener, deodorant cream, deodorant preparations for absorbing animal odours, deodorant preparations for absorbing cooking odours, deodorant preparations for household use, deodorant preparations, cosmetic anti-ageing cream, ultra violet light (UV) sun blocking preparation, cosmetic anti-wrinkle cream, detergent having anti-bacterial properties, herbal anti-dandruff shampoo, medicated soap, footwear deodorising spray, antiseptic cleanser, skin cleanser, agricultural disinfectant, cleaning cloths impregnated with disinfectant for hygiene purposes, detergents for medical use having disinfectant properties, disinfectant dressings, disinfectant swabs, disinfectant washes, disinfectant, impregnated towels containing disinfectant preparations, oral disinfectants, sponges impregnated with disinfectant, anti-bacterial pharmaceutical preparations, anti-bacterial preparations for veterinary use, anti-inflammatory agents, anti-inflammatory preparations, anti-microbial preparations, compounds having an anti-inflammatory activity, skin lightening products, detergents for medical use having anti-bacterial properties, herbal anti-dandruff preparations, medicated anti-bacterial face washes, germicidal cleaners, germicidal preparations, germicidal preparations for personal use, medicated cleaning preparations, medicated cleansing preparations for the skin, medicated cream for use in washing, medicated soap, non-soapy cleansing preparations for medical use, inner soles for footwear, athletic footwear.

### BRIEF DESCRIPTION OF THE DRAWINGS

One or more embodiments of, or tests and processes relating to, the present invention will hereinafter be described with reference to the accompanying Figures, in which:

FIG. 1 shows a chart of growth inhibitory activity of T. ferdinandiana leaf (L) and fruit (F) extracts against C. jeikeium measured as zones of inhibition (mm).

FIG. 2 shows a chart of growth inhibitory activity of T. ferdinandiana leaf (L) and fruit (F) extracts against S. epidermidis clinical isolate measured as zones of inhibition (mm).

FIG. 3 shows a chart of growth inhibitory activity of T. ferdinandiana leaf (L) and fruit (F) extracts against P. acnes measured as zones of inhibition (mm).

FIG. 4 shows a chart of growth inhibitory activity of T. ferdinandiana leaf (L) and fruit (F) extracts against B. linens measured as zones of inhibition (mm).

In the test results shown in FIGS. 1 to 4: F=fruit; L=leaf; M=methanolic extract; W=aqueous extract; E=ethyl acetate extract; C=chloroform extract; H=hexane extract; Van=vancomycin (5 μg). Results are expressed as zones of inhibition±SEM.

FIG. 5 shows a chart of the lethality of the T. ferdinandiana fruit and leaf extracts (2000 μg/mL) and the potassium dichromate (1000 μg/mL) and seawater controls towards Artemia franciscana nauplii (brine shrimp) after 24 hours exposure.

In the test results shown in FIG. 5: F=fruit; L=leaf; M=methanolic extract; W=aqueous extract; E=ethyl acetate extract; C=chloroform extract; H =hexane extract; PC=potassium dichromate control; NC=negative (seawater) control. Results are expressed as mean % mortality±SEM.

FIG. 6 shows two charts, a and b, chart a showing positive and chart b showing negative ion RP-HPLC compound chromatograms (TCC) of 24 injections of T. ferdinandiana leaf methanolic extract.

FIG. 7 shows a table of results for qualitative LC-MS analysis of T. ferdinandiana leaf extract, elucidation of empirical formulas and putative identification of tannins and other major compounds.

### DESCRIPTION OF PREFERRED EMBODIMENT

Study and testing found that leaf extracts from T. ferdinandiana T. ferdinandiana were more potent bacterial growth inhibitors than fruit extracts from the same plant.

The leaf methanolic extract was found to be particularly potent, with low minimum inhibitory concentration (MIC) values against Corynebacterium jeikeium, Staphylococcus epidermidis, Propionibacterium acnes and Brevibacterium linens.

The aqueous and ethyl acetate leaf extracts of T. ferdinandiana were also found to be potent growth inhibitors of Corynebacterium jeikeium, Staphylococcus epidermidis.

In comparison, T. ferdinandiana fruit extracts were substantially less potent antibacterial agents, although still with MIC values indicative of growth inhibitory activity.

Non-biased phytochemical analysis of the methanolic leaf extract revealed the presence of high levels of and high diversity of tannins and high levels of the flavone luteolin.

Tests:

A mass of 1 g of powder of coarsely ground T. ferdinandiana leaf was extensively extracted with 50 mL of either methanol, deionised water, ethyl acetate chloroform or hexane or for 24 hours at 4° C. with gentle shaking.

All solvents were AR grade (standard Mallinckrodt grade of analytical reagents; suitable for laboratory and general use.)

The extracts were filtered through filter paper (Whatman No. 54) and air dried at room temperature.

The aqueous extract was lyophilised by rotary evaporation in an Eppendorf concentrator 5301.

The resultant pellets were dissolved in 10 mL deionised water containing 0.5% dimethyl sulfoxide (DMSO) and then passed through a 0.22 μm filter (Sarstedt) and stored at 4° C. until used.

Evaluation of antibacterial activity screening of the T. ferdinandiana fruit and leaf extracts was assessed using a modified disc diffusion assay.

100 μL of each individual bacteria (C. jeikeium, B. linens, P. acnes and S. epidermidis) was grown separately in 20 mL of the appropriate broth until an approximate count of 10⁸ cells/mL was reached.

A volume of 100 μL of each bacterial suspension was spread onto agar plates and the extracts tested for antibacterial activity using 5 mm sterilised filter paper discs.

B. linens, P. acnes and S. epidermidis cultures were spread onto respective nutrient agar plates.

C. jeikeium cultures were spread onto nutrient agar plates supplemented with 300 μL Tween 80/L of agar.

Discs were infused with 10 μL of the individual extracts, allowed to dry and placed onto inoculated plates. The plates were left to stand at 4° C. for 2 hours before incubation.

Plates inoculated with B. linens, S. epidermidis or C. jeikeium cultures were incubated aerobically at 37° C. for 24 hours.

Plates spread with P. acnes cultures were incubated under induced anaerobic conditions for 37° C. for 72 hours.

The diameters of the inhibition zones were measured to the closest whole millimetre. Each assay was completed in at least triplicate. Mean values (±SEM) are reported below.

Ampicillin (10 μg) and vancomycin (5 μg) discs were used as a positive control to compare antibacterial activity, and filter discs infused with 10 μL of distilled water were used as a negative control.

MIC for each extract was determined using two methods:
A liquid dilution MIC assay was used for its sensitivity; and
A microplate liquid dilution MIC assay was used because of it being a common method of quantifying bacterial growth inhibition efficacy.

A solid phase agar disc diffusion assay was also conducted as representative of the environment and conditions relevant to solid axillary and foot skin systems.

The methanolic extracts of T. ferdinandiana leaf displayed the greatest potency in all bacterial growth inhibition assays mentioned above. The methanolic extracts are therefore considered the most effective candidates.

In total, 145 unique mass signals were noted for methanolic T. ferdinandiana leaf extracts.

The ability of T. ferdinandiana leaf and fruit extracts to inhibit the growth of bacterial species under test associated with the production of body and foot odours were assessed. 10 μL of each extract was screened using a disc diffusion assay.

Fruit to leaf extract proportions may vary dependent upon the type of product and purpose for the product.

Proportions may vary between 15 fruit extract to 99% leaf extract, to 99% fruit extract to 1% leaf extract, preferably between 5% fruit extract to 95% leaf extract.

Other components may be provided in the composition in that the composition need not be entirely fruit and leaf extract but can include a carrier material (such as a base cream or lotion), an active material (such as an active skin lightening agent or a barrier agent (e.g. SPF factor sunblock/zinc based product).

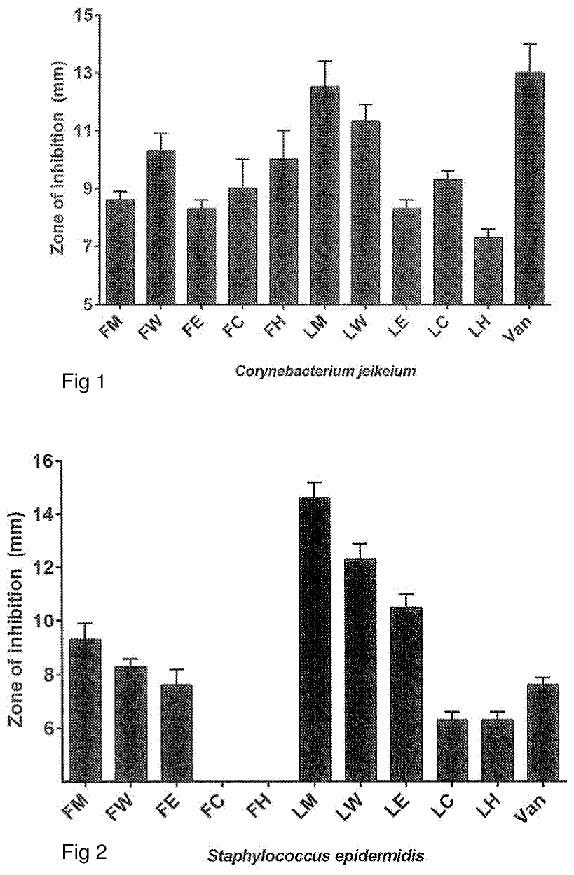

C. jeikeium growth was inhibited by all of the T. ferdinandiana leaf and fruit extracts screened—see FIG. 1.

Whilst the leaf and fruit extracts were both good inhibitors of C. jeikeium growth, the leaf extracts were generally more potent inhibitors.

The methanolic and aqueous leaf extracts were particularly potent inhibitors.

The growth inhibitory activity of all of the T. ferdinandiana fruit and leaf extracts assayed compared favourably with the antibiotic controls. Whilst vancomycin was a good inhibitor of C. jeikeium growth, it was no more potent than the methanolic T. ferdinandiana leaf extract. For reference, the vancomycin control consisted of a relatively high does of a pure antibiotic, whilst the extracts are mixtures of several individual compounds.

As C. jeikeium contributes strongly to axillary malodour, and also contributes to foot odour formation, the T. ferdinandiana extracts (particularly the methanolic leaf extract) were found to be the most suitable for use in personal hygiene products aimed at controlling or eliminating axillary malodour and/or foot malodour.

It has been realised that, when C. jeikeium growth is inhibited, there is less competition for other malodour producing bacteria and these may increase in the absence of C. jeikeium. Thus, it would be beneficial for personal hygiene products aimed at controlling axillary malodour and/or foot malodour producing bacteria to also inhibit the growth of other bacteria, such as Staphylococcus spp. and Propionibacterium spp.

As Staphylococcus spp. and Propionibacterium spp. contribute to axillary malodour formation, and Propionibacterium spp. and Brevibacterium spp. contribute to foot malodour, and effective deodorant would need to also inhibit the growth of these bacteria.

Notably, the growth of S. epidermidis was highly susceptible to some T. ferdinandiana fruit and leaf extracts (see FIG. 2). Consistent with the trend noted for C. jeikeium growth inhibition, S. epidermidis was also more susceptible to the T. ferdinandiana leaf extracts than to the fruit extracts.

Again, the high polarity methanolic and aqueous extracts were the best growth inhibitors, indicating that the antimicrobial components of the extracts were polar.

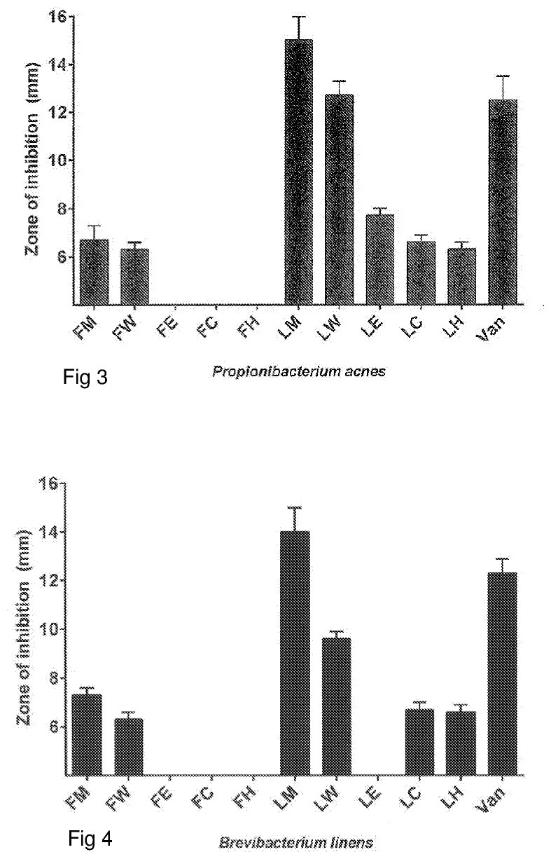

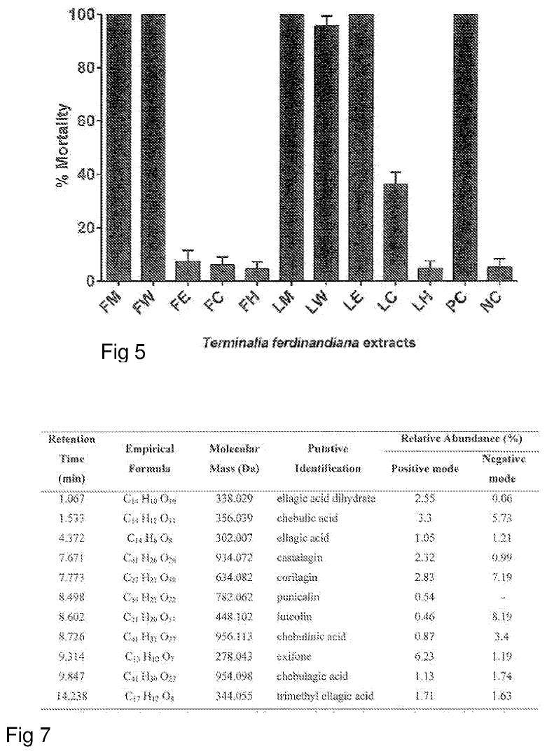

Similarly, the growth of P. acnes was also strongly inhibited by several of the T. ferdinandiana leaf extracts—see FIG. 3.

The polar to mid polarity T. ferdinandiana leaf extracts were generally substantially more potent inhibitors of P. acnes growth than for C. jeikeium and S. epidermidis.

As evident for the growth of C. jeikeium and S. epidermidis, the leaf extracts were generally more potent bacterial growth inhibitors than were the fruit extracts, and the methanolic and aqueous extracts had the greatest efficacy.

The ability of the T. ferdinandiana fruit and leaf extracts to inhibit B. linens growth was also tested—see FIG. 4.

B. linens was particularly susceptible to the methanolic leaf extract. Good inhibition was also recorded for the aqueous leaf extract.

The B. linens growth inhibitions by the methanolic and aqueous leaf extracts were particularly potent compared to that of the ampicillin control.

As the aforementioned tests revealed that the methanolic leaf extract of T. ferdinandiana provides the most effective bacterial growth inhibitory activity, phytochemical analysis was carried out.

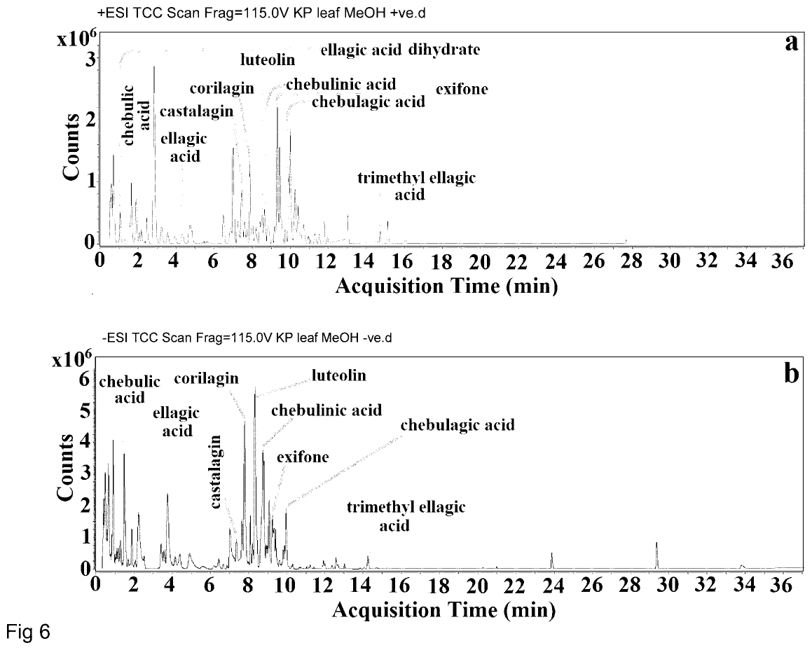

Charts a and b in FIG. 6 show count (×10⁶) v Acquisition Time (min). The table in FIG. 7 identifies relative abundance of as range of named active components in the extract.

145 compounds were detected in non-biased liquid chromatography-mass spectrometry (LC-MS) analysis, and 85 of those 145 compounds were putatively identified.

11 of the 85 compounds were identified as tannins, being a major component of the extract and a wide diversity of tannins.

Chebulic acid, corilagen, chebulinic acid and chebulagic acid chromatographic peaks accounted for approximately 6%, 7%, 3% and 2% respectively of the total peak areas for negative ionisation mode chromatogram—See chart b of FIG. 6 and also the table in FIG. 7.

Ellagic acid and its derivatives account for a further 3% of the relative abundance of methanolic T. ferdinandiana leaf extract peak areas in the negative ionisation mode (FIG. 6 chart b).

In addition, 4-galloylpyrogallol (exifone) and castalgin were estimated to be approximately 6% and 3% of the total compounds in the positive ionisation mode respectively.

The broad range of active compounds within the T. ferdinandiana leaf extract provides a symbiotic activity beyond the efficacy of any one of those compounds.

Where one, or even several, compounds may be effective in controlling/inhibiting growth of one type of bacteria, the combination of compounds not only individually inhibit specific types of bacteria but also have a combined effect on the range of bacteria.

The T. ferdinandiana extract containing or being extract of T. ferdinandiana leaf may can be incorporated into or be a skin care and/or personal hygiene product, such as a deodorant cream, lotion, gel, liquid or spray.

1. A composition for use in inhibiting growth of bacteria, the composition including extract of Terminalia ferdinandiana (T. ferdinandiana) leaf.

2. The composition according to claim 1, wherein the composition inhibits the growth of one or more species of bacteria selected from Corynebacterium spp., Staphylococcus spp., Propionbacterium spp., and Brevibacterium.

3. The composition according to claim 2, wherein the composition inhibits growth of one or more of Corynebacterium jeikeium, Staphylococcus epidermidis, Propionibacterium acnes and Brevibacterium linens.

4. The composition according to claim 1, included in or provided as a personal hygiene product.

5. The composition according to claim 4, provided as a personal hygiene product to control axillary malodour and/or foot malodour.

6. The composition according to claim 1, including T. ferdinandiana fruit extract.

7. The composition according to claim 1, provided as, or in, a cream, lotion or gel.

8. The composition according to claim 1, provided as, or in, a solid, liquid or spray product for application to skin.

9. The composition according to claim 1, the composition provided in or as a personal deodorant or footcare product.

10. The composition according to claim 1, wherein the composition is provided as a surface preparation or surface treatment product to control bacterial growth.

11. The composition according to claim 1, the composition incorporated into one or more of a liquid cleanser, anti-bacterial additive for liquids, antimicrobial preservative for cosmetics, antimicrobial preservative for pharmaceuticals, personal hygiene products, cleanser for the face; cosmetic acne cleanser, facial cleanser, hand cleanser, skin lightening products, detergent having disinfectant properties, disinfectant soap, disinfectant soap solution, detergent having disinfectant properties.

12. The composition according to claim 1, the composition incorporated into one or more of an antiperspirant deodorant, body deodorant, deodorant cream for personal use, deodorant preparation for personal use, deodorant soap; deodorant spray for personal use, deodorant sticks for personal use, deodorant for human beings or for animals, deodorant for personal use; deodorant for pets; personal body deodorant, foot deodorant, douching preparation for personal sanitary or deodorant purposes, essential oils for use in deodorants, perfumed deodorant for personal use, roll-on deodorant, scented deodorant preparations for personal use, deodorant for clothing and textiles, deodorant for household use, perfumed deodorant, room air freshener, deodorant cream, deodorant preparations for absorbing animal odours, deodorant preparations for absorbing cooking odours, deodorant preparations for household use, deodorant preparations.

13. The composition according to claim 1, the composition incorporated into one or more of a cosmetic anti-ageing cream, ultra violet light (UV) sun blocking preparation, cosmetic anti-wrinkle cream.

14. The composition according to claim 1, the composition incorporated into one or more of a detergent having anti-bacterial properties, herbal anti-dandruff shampoo, medicated soap, footwear deodorising spray, antiseptic cleanser, skin cleanser, agricultural disinfectant, cleaning cloths impregnated with disinfectant for hygiene purposes, detergents for medical use having disinfectant properties, disinfectant dressings, disinfectant swabs, disinfectant washes, disinfectant, impregnated towels containing disinfectant preparations, oral disinfectants, sponges impregnated with disinfectant, anti-bacterial pharmaceutical preparations, anti-bacterial preparations for veterinary use, anti-inflammatory agents, anti-inflammatory preparations, anti-microbial preparations, compounds having an anti-inflammatory activity, skin lightening products, detergents for medical use having anti-bacterial properties, herbal anti-dandruff preparations, medicated anti-bacterial face washes, germicidal cleaners, germicidal preparations, germicidal preparations for personal use, medicated cleaning preparations, medicated cleansing preparations for the skin, medicated cream for use in washing, medicated soap, non-soapy cleansing preparations for medical use, inner soles for footwear, athletic footwear.

15. The composition according to claim 1, wherein the leaf extract includes one or more of a methanolic extract, aqueous extract; ethyl acetate extract; chloroform extract; or hexane extract of the T ferdinandiana leaf.

16. The composition according to claim 1, wherein the leaf extract includes a proportion of at least one tannin.

17. The composition according to claim 16, wherein the at least one tannin includes one or more of chebulic acid, corilagen, chebulinic acid and chebulagic acid.

18. The composition according to claim 1, wherein the leaf extract includes at least one flavone.

19. The composition according to claim 18, the at least one flavone including luteolin.

20-21. (canceled)

22. A method of inhibiting growth of bacteria includes applying a composition containing an extract of Terminalia ferdinandiana (T. ferdinandiana) leaf to a surface having bacteria thereon or a surface at risk of having bacteria thereon.

23. The method according to claim 22, wherein the surface is human skin and the composition is applied to the skin.

24. The method according to claim 23, wherein the composition is applied to axillary regions and/or at least one foot of a person.

25. The method according to claim 23, wherein the composition is applied by spray or application of a gel, lotion or cream.

26. An extract of T. ferdinandiana including extract of T. ferdinandiana leaf, the extract for use as a bacteria inhibition composition.

27. The extract of T. ferdinandiana according to claim 26, further including extract of T. ferdinandiana fruit.

28. The extract of T. ferdinandiana according to claim 26, including at least one tannin and/or at least one flavone.

29. The extract of T. ferdinandiana according to claim 28, wherein the at least one tannin includes one of or a combination of two or more of, chebulic acid, corilagen, chebulinic acid and chebulagic acid.

30. The extract of T. ferdinandiana according to claim 28, including at least one flavone.

31. The extract of T. ferdinandiana according to claim 30, wherein the at least one flavone includes luteolin.

32. The extract of T. ferdinandiana leaf according to claim 26, incorporated into one or more of a liquid cleanser, anti-bacterial additive for liquids, antimicrobial preservative for cosmetics, antimicrobial preservative for pharmaceuticals, personal hygiene products, cleanser for the face; cosmetic acne cleanser, facial cleanser, hand cleanser, skin lightening preparations, detergent having disinfectant properties, disinfectant soap, disinfectant soap solution, detergent having disinfectant properties.

33. The extract of T. ferdinandiana leaf according to claim 26, incorporated into one or more of an antiperspirant deodorant, body deodorant, deodorant cream for personal use, deodorant preparation for personal use, deodorant soap; deodorant spray for personal use, deodorant sticks for personal use, deodorant for human beings or for animals, deodorant for personal use; deodorant for pets; personal body deodorant, foot deodorant, douching preparation for personal sanitary or deodorant purposes, essential oils for use in deodorants, perfumed deodorant for personal use, roll-on deodorant, scented deodorant preparations for personal use, deodorant for clothing and textiles, deodorant for household use, perfumed deodorant, room air freshener, deodorant cream, deodorant preparations for absorbing animal odours, deodorant preparations for absorbing cooking odours, deodorant preparations for household use, deodorant preparations.

34. The extract according to claim 26, incorporated into one or more of a cosmetic anti-ageing cream, ultra violet light (UV) sun blocking preparation, cosmetic anti-wrinkle cream.

35. The extract according to claim 26, incorporated into one or more of a detergent having anti-bacterial properties, herbal anti-dandruff shampoo, medicated soap, footwear deodorising spray, antiseptic cleanser, skin cleanser, agricultural disinfectant, cleaning cloths impregnated with disinfectant for hygiene purposes, detergents for medical use having disinfectant properties, disinfectant dressings, disinfectant swabs, disinfectant washes, disinfectant, impregnated towels containing disinfectant preparations, oral disinfectants, sponges impregnated with disinfectant, anti-bacterial pharmaceutical preparations, anti-bacterial preparations for veterinary use, anti-inflammatory agents, anti-inflammatory preparations, anti-microbial preparations, compounds having an anti-inflammatory activity, detergents for medical use having anti-bacterial properties, herbal anti-dandruff preparations, medicated anti-bacterial face washes, germicidal cleaners, germicidal preparations, germicidal preparations for personal use, medicated cleaning preparations, medicated cleansing preparations for the skin, medicated cream for use in washing, medicated soap, non-soapy cleansing preparations for medical use, inner soles for footwear, athletic footwear.
